(19) United States
(12) Patent Application Publication
(10) Pub. No.: US 2020/0246526 A1
(43) Pub. Date: Aug. 6, 2020

(54) DEVICE AND METHOD FOR CONNECTING TWO BLOOD VESSEL SECTIONS

(71) Applicant: Berlin Heart GmbH, Berlin (DE)

(72) Inventors: Ares K. Menon, Berlin (DE); Thomas Wöhling, Seevetal (DE); Eugen Sandica, Bad Oeynhausen (DE)

(73) Assignee: Berlin Heart GmbH, Berlin (DE)

(21) Appl. No.: 16/487,857

(22) PCT Filed: Feb. 22, 2018
(86) PCT No.: PCT/EP2018/054392

§ 371 Date: Aug. 22, 2019

(30) Foreign Application Priority Data
Feb. 22, 2017 (EP) 17157481.7

(51) Int. Cl. A61M 1/12; A61M 1/10; A61M 1/36

(52) U.S. Cl. CPC A61M 1/125; A61M 1/122; A61M 1/3659; A61M 1/1008

(57) ABSTRACT

A surgical intervention is often required in patients with failing Fontan circulation in order to stabilise the patients prior to undergoing a heart transplant. Said type of stabilisation can be carried out using a system described herein comprising a device with two inlet openings and an outlet opening.

### CROSS-REFERENCE TO RELATED APPLICATIONS

This application is a 371 nationalization of international patent application PCT/EP2018/054392 filed Feb. 22, 2018, which claims priority under 35 USC § 119 to European patent application EP17157481.7 filed Feb. 22, 2017. The entire contents of each of the above-identified applications are hereby incorporated by reference.

### BRIEF DESCRIPTION OF THE DRAWINGS

FIG. 1 shows a starting state prior to an embodiment of the method for implantation of a device;

FIG. 2 shows an already pre-implanted device with schematically shown pump connections;

FIG. 3A shows a first embodiment of a device;

FIG. 3B shows a cross section of a first embodiment of a device;

FIG. 4A show a further embodiment of a device;

FIG. 4B shows a cross section of a further embodiment of a device;

FIG. 5A show a further embodiment of a device;

FIG. 5B shows a cross section of a further embodiment of a device;

FIG. 6 show a system with a device according to FIG. 5;

FIG. 7A show a further embodiment of a device;

FIG. 7B shows a cross section of a further embodiment of a device; and

FIG. 8 shows a depiction of the connection of the cannulas to the body's own heart/vascular system.

### DETAILED DESCRIPTION

The present application relates to a device for connecting two blood vessel sections, and a use of a device of this kind, and a method for implanting a device of this kind.

In the treatment of congenital heart defects, what are known as “single-ventricle” diseases require quick correction in the sense of a palliation. In the field of medicine, a “single ventricle” heart refers to a condition of congenital heart defects in which only one functioning ventricle has formed during foetal development. The clinical picture comprises a wide range of forms, which also have different effects on the circulation system and the therapeutic approach. A common feature of all forms is that only one ventricle actually exists, or one primary chamber, which receives its blood from both atria, with a secondary chamber, which receives its blood from the primary chamber. A distinction is made here between two basic forms depending on whether the right ventricle or the left ventricle has not formed sufficiently.

The surgical therapy in the form of palliative interventions and an operation which improves the situation is dependent on the exact anatomical constellation and is decided for each patient, in particular for each child, individually. In the case of defects of the right ventricle, particularly with regard to the closure of the tricuspid and/or pulmonary valve, a Fontan operation is selected. In the case of defects of the left ventricle, in particular a hypoplastic left heart syndrome, a Norwood operation is recommended.

In the case of the Fontan operation the actual absence of one ventricle is compensated for, usually in three surgical steps, by the establishment of an individual “system chamber”, which supplies the main circulation system of the body, and the establishment of a passive pulmonary circulation. The sole, normally large ventricle is in this case usually the right ventricle, which is naturally weak. This must supply the circulation system of the body, whereas the blood flows merely passively via the large veins of the body (venae cavae) into and through the lungs, without a chamber actively pumping this venous blood into the lungs. The system is referred to as a “Fontan circuit” or total cavo-pulmonary connection (TCPC for short).

Typically, patients with this circuit form suffer from two primary problems: firstly, the failure of the passive pulmonary circulation and/or secondly the failure of the then single “system ventricle”. In the first case, the lack of active pumping power leads to a venous backlog in the lower half of the body with water retention (oedemas), abdominal dropsy (ascites), blockages in abdominal organs such as the liver, kidneys and intestine with severe functional impairment accordingly (protein-loss syndrome, kidney and liver failure, etc.). In the second case the system ventricle wears out—usually after two to three decades—and insufficient pumping is experienced. The right heart muscle, which is naturally weak, is not powerful enough for lifelong supply of the main circulation system of the body.

A heart transplant is often the only possible therapy option. However, since the starting state of the patient with deteriorating circulation (what is known as “failing Fontan” circulation) is worse than in other organ receivers, the success rate following a heart transplant is poor. One of the problems is the lack of systematic conditioning of the patient with failing Fontan circulation. There is a need for instruments that make it possible in the case of patients with failing Fontan circulation to improve the basic state, i.e. to achieve a stabilisation of the circulation system prior to undergoing a heart transplant.

Besides the heart defects already discussed, the device described in this application and a corresponding method for use of the device can also be employed in the therapy of other congenital heart defects, for example heart defects that require the temporary use of a “total artificial heart” until a donor heart is available or usable.

In the prior art, some techniques for improving the circulation prior to undergoing a heart transplant are known. In US 2004/0147803 a system with an inflatable sheath is proposed, wherein the sheath is placed around the superior vena cava (SVC) or the inferior vena cava (IVC). By rhythmically inflating and deflating the sheath, the vena cava is stimulated and an additional pumping power is imparted to the blood of the vena cava, so that the pulmonary circulation is supported.

In EP 2 341 956 A1 a system is proposed in which a pump for supporting the pulmonary circulation is placed directly in the SVC or IVC and actively pumps blood into the pulmonary veins in the scope of a Fontan anastomosis.

The object of the present invention is to provide an alternative to the above-mentioned devices and methods. The object is achieved in accordance with the provisions of the accompanying claims.

In accordance with the application a device for connecting two blood vessel sections, preferably the end of the SVC and an end of the IVC, is provided. The device comprises a fluid-tight hollow body with a first end section, a second end section and an intermediate section arranged between the first and second end section.

The hollow body thus creates a direct connection between the two blood vessel sections and is inserted as a prosthesis so to speak between the two blood vessel sections. The first and/or the second end section can be formed such that they are connected directly to the ends of the respective blood vessel sections. Alternatively, a further material section can be arranged at the first and/or second end section, wherein the further material section is connected to the blood vessel.

The hollow body is fluid-tight, i.e. liquid flowing through between the first and the second end section can escape substantially only through openings in the hollow body. To this end, a first opening is provided in the first end section and a second opening is provided in the second end section. Liquid, for example blood, is thus prevented from escaping outwardly through the hollow body or a wall of the hollow body and from gushing into the abdominal cavity.

Besides the first and the second opening, the intermediate section has a third opening. The third opening is used to drain the blood flowing in through the first and/or the second end section. The intermediate section, in the region of the third opening, has a coupling device for a blood pump or a blood cannula which is connected to a blood pump.

As a result of the presence of a coupling device in the region of the third opening, a blood pump can be connected either directly to the coupling device or to a blood cannula arranged on the coupling device, following the implantation of the device according to the application, and a process of active pumping of the blood flowing from the two blood vessel sections can thus be supported by a pump. Here, the device can be part of a system which, besides the device according to the application, also comprises a blood pump for attachment to the device. The blood pump can be an extracorporeal or an intracorporeal heart assist pump. The coupling device for the blood pump or the blood cannula attached to the coupling device can be directly coupled to the inlet of the blood pump or heart assist pump, and the outlet of the pump can be attached for example to the pulmonary veins by means of a further cannula or a graft.

The pumps can be, for example, an EXCOR® pump or an INCOR® pump from Berlin Heart GmbH, a pump from the HeartMate range by Thoratec, an MVAD or a short-term heart pump, as are used in the field of ECMO therapies. Further intracorporeal or extracorporeal heart assist systems obtainable on the market can also be used in the system.

By way of the device it is essentially made possible to attach a blood pump for assisting pulmonary circulation directly to the circulation system. The relief of the circuit necessary for the patient is hereby implemented reliably, and the patient can rest reliably prior to an upcoming heart transplant.

Further exemplary embodiments can be found in the dependent claims and in the following explanations.

In some embodiments the first and the second opening are substantially opposite one another or their surface normals of the flow areas are substantially coaxial. The third opening or surface normal thereof runs substantially at an angle between 60° and 120°, preferably between 75° and 105°, to the other surface normals.

In one embodiment the coupling device is connected integrally to the intermediate section. This means for example that the coupling device is preferably produced in one piece with the intermediate section, i.e. that the intermediate section and the coupling device are made of the same material. The handling of the device at the time of attachment of the device to a blood pump is hereby simplified. Alternatively, however, the term “integrally” is understood to mean that the coupling device is not connected to the intermediate section in one piece, but instead fixedly and permanently. For example, the coupling device can be sewn, welded or adhesively bonded to the intermediate section. In some embodiments it would appear to be simpler to connect the coupling device to the intermediate section only following the production of the device.

In a further embodiment the coupling device is a cannula, preferably a silicone cannula. The cannula can be made such that it has a sufficient length to be connected to an extracorporeal heart assist pump. This can mean in particular that the cannula can have a length between 40 and 100 cm. In this embodiment it can be advantageous that the length of an operation can be kept as short as possible and that additional connection pieces between a heart assist pump and the device are spared. There is thus a higher likelihood that no blood clots will form.

Silicone may be a preferred material here for construction of the cannula. Silicone is biocompatible and can have good rigidity properties depending on its wall thickness. In particular, silicone cannulas appear to be well suited for counteracting the suction pressure of a pump without the interior of the cannula collapsing. However, the cannula can also be made of other biocompatible materials.

In a further embodiment the coupling device is a suture ring. In this embodiment a suture ring, as is used for example in intra-corporeal heart assist systems at the apex of the heart, can be arranged on the device in the region of the third opening or running around the third opening. The blood pump can then be connected via its inlet to the suture ring, and a fluid-tight connection between the heart assist system and the device can thus be produced. The blood pump can be inserted directly into the suture ring or can be connected to the device via a cannula.

In a further embodiment the device comprises, as coupling device, an adapter for attachment of a cannula or a suture ring to a blood pump. The adapter can be made such that a conventional cannula for an extracorporeal system can be fitted onto the adapter.

For example, the approach adopted in a Luer lock system can be employed here similarly.

In a further embodiment the first and the second end section are formed such that they can be sewn or connected to a blood vessel via a connector, i.e. can be connected in a fluid-tight manner. For connection by way of a connector, a connector can be provided for example by means of a thread lacing, cable tie, or a sheath. The first and the second end section can thus be made of different materials as compared to the intermediate section. For example, a material of the first or the second end section can be selected such that it has a thinner wall thickness and therefore can be sewn or otherwise connected more easily to a vessel. Exemplary materials are Dacron® (DuPont), an alternative polyethylene glycol terephthalate or ePTFE (expanded polytetrafluoroethylene). In the case of a fluid-tight connection by means of a connector, the first and/or second end section can also comprise a metal or biocompatible plastic in order to provide the connector selected for the connection with a counter force, so that the end of the blood vessel lying between the connector and the first or second end section can be fixedly coupled to the device.

Alternatively or additionally, the first and/or second end section can comprise a collar which has another, more easily sewable material or a smaller wall thickness of the same material of the intermediate section as compared to the material of the intermediate section. The material is made for example in such a way that the collar can be easily trimmed using scissors or a scalpel. For example, the collar can be made of a textile material, for example a graft material, or can comprise such material. The collar or the collars is/are formed in such a way that an easier connection (for example sewing) of the end sections to the vessels or the remaining material of the Fontan tunnel is made possible. Furthermore, the collar can be shortened after the connection, so that the length of the device between the first and second end section can be adapted to the patient. For example, the length of the one collar (precisely one) or the collars (more than one, for example two collars) can lie between 1 cm and 5 cm. The collar can be connected integrally to the material of the intermediate section or for example can be sewn or adhesively bonded thereto.

In a further embodiment the hollow body is formed in one piece, i.e. the first and the second end section and the intermediate section made of the same material. This for example can simplify the production of the device and can prevent the potential formation of blood clots in the region of the transitions between end section and intermediate section.

In a further embodiment the hollow body has a cross-section that increases from the first end section to the intermediate section and decreases again from the intermediate section to the second end section. This “bulged” embodiment of the hollow body causes the blood flowing in through the first and the second blood vessel to enter an enlarged volume before it is sucked through the third opening into the inlet of a heart pump. This additionally relieves the pressure of the vessels connected to the device. Furthermore, by changing the wall thickness in some areas of the hollow body, for example in the intermediate region, an easier extensibility of the hollow body in these regions can be achieved (“compliance”).

In the bulged embodiments, the ratio of the flow area F_1 and F_2 of the first and second opening respectively to the flow area F_B at the “most bulged” point between F_1/F_B can be <1 or F_1/F_B can be <0.9 or F_1/F_B<0.8. In some exemplary embodiments the wall thickness in the region of the first and second opening is substantially the same as the wall thickness of the most bulged point. Alternatively, the wall thickness in the region of the most bulged point can be less than in the region of the intermediate and end sections.

In a further embodiment the bulged embodiment of the hollow body is asymmetrical. This can mean that the cross-section does not increase in all directions around a body axis of the hollow body, but merely in the directions of an angle by less than or equal to 180° around the body axis. In this embodiment the bulged half of the hollow body can be arranged in the body in such a way that the bulge comes to rest at the position of the, for example, right atrium.

In a further embodiment the hollow body is such that it has a length between the first and the second end section between 3 and 15 cm. In this way, the conditions within the human body are taken into consideration, but at the same time it is ensured that the pump is reliably connected to the coupling device, without exerting additional stress on the blood vessel ends.

Alternatively or additionally to the embodiments above, the flow areas of the first and second opening are larger than the flow area of the third opening. A flow area is understood to mean the area, perpendicular to the blood flow direction, that is free and clear as a result of the opening. Here, the flow area of the first opening can be between 1.5 cm² and 3.5 cm² (the first opening is formed in such a way that it is connectable to the superior vena cava, wherein the vena cava for example has a diameter between 14-20 mm), the flow area of the second opening can be between 2.5 cm² and 4.5 cm² (the second opening is formed in such a way that it is connectable to the superior vena cava, wherein the vena cava for example has a diameter between 18-24 mm). Provided the opening is substantially circular, the corresponding diameter or radius of the flow area can also be determined. The third opening has a smaller flow area than the first and/or second opening and can lie between 0.5 cm² and 1.5 cm², but can also be larger (for example up to 4 cm²), provided no cannula is used and instead a pump is inserted directly into the third opening. Alternatively, the ratio of the flow area F_3 of the smaller third opening to the flow areas F_1 and F_2 of the larger openings can be described as F_3/F_1<1, preferably F_3/F_1<0.8, particularly preferably as F_3/F_1<0.6.

The system or device presented here can be used in a patient with failing Fontan circulation to ensure assistance of the heart prior to a heart transplant, which causes the vascular system of the patient to be better prepared for the upcoming transplant.

The invention will be explained in greater detail hereinafter with reference to a number of figures.

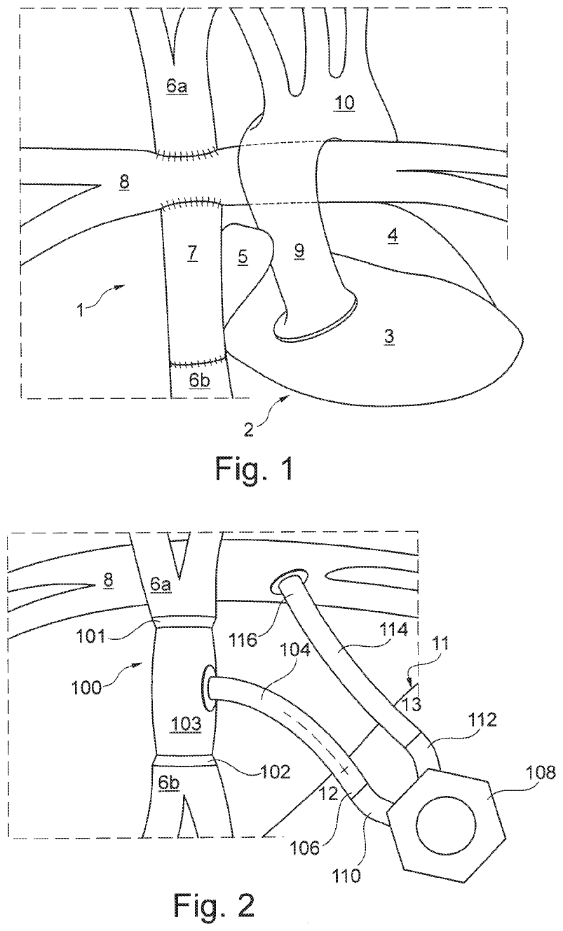

With reference to FIG. 1, a possible starting position prior to the implantation of the device according to the application will be presented. What is shown here is the cavopulmonary anastomosis proposed by Marceletti in 1988, wherein the connection between the superior and the inferior vena cava to the right atrium has been separated. A detail of the circulatory system 1 with a heart 2 that has merely one ventricle 3, a left atrium 4 and a right atrium 5 is shown in FIG. 1. The heart valve between the ventricle 3 and the right atrium 5 has been permanently closed, so that the ventricle has only a connection to the left atrium. The superior vena cava (SVC) 6a and a Fontan tunnel 7 have been connected to the pulmonary artery 8 by means of a suture. The Fontan tunnel 7n has been sewn at its lower end to the inferior vena cava (IVC) 6b and can be, for example, a prosthesis manufactured from PTFE. Blood thus flows passively from the vessels 6a , 6b and 7 into the pulmonary arteries through the lungs and then into the left atrium 4. The blood is transported from the left atrium 4 into the ventricle 3 and is then transported via the aorta 9 into the aortic arch 10 and from there into the various arteries of the body.

Alternatively, the lower and the upper venae cavae can also be connected furthermore to the right atrium, wherein the heart valve to the ventricle is closed and the entry to the pulmonary artery is arranged in the right atrium. An approach of this kind will also create a passive circuit to the lungs.

Proceeding from the exemplary embodiment of FIG. 1, a device according to the application can now be connected to the circulatory system 1.

To this end, the SVC 6a and the Fontan tunnel 7 are separated from the pulmonary artery 8, the Fontan tunnel is removed or cut back so that an edge of the Fontan tunnel remains for connection of the device (for example by sewing the device to the remaining edge of the Fontan tunnel or by connecting the device by way of a connector to the remaining edge of the Fontan tunnel), and then, as shown in FIG. 2, a device is sewn to the ends of the SVC 6a and the IVC 6b (in the embodiment of the method which leads to the state shown in FIG. 2, the Fontan tunnel has been removed). As a result of the device, blood can now flow from the SVC 6a and the IVC 6b from top to bottom into the device. The hollow body 100 of the device, besides the end sections 101 and 102 sewn to the vessels, also has an intermediate section 103, which integrally comprises a coupling device 104 in the form of a cannula. The cannula in this case is guided out from the body of the patient and penetrates the skin 11 at the point 12, for example beneath the rib cage. The end 106 of the cannula 104 which serves as coupling device is connected to a blood pump 108. The inlet 110 of the blood pump 108 is coupled to the cannula 104 via the end 106 thereof. The outlet 112 of the pump is connected to a further cannula 114, wherein the end of the cannula 114 not connected to the pump is attached directly to the pulmonary artery. To this end, the end 116 of the cannula 114 for example can be sewn to the pulmonary artery 8. In this way, an active supply (by means of the blood pump 108) of the pulmonary artery with blood from the SVC 6a and the IVC 6b is ensured and therefore the heart 2 (not shown in greater detail in FIG. 2) is considerably relieved. In particular, the vessels, such as the SVC and the IVC, can relax before a donor heart is introduced.

Various embodiments of the device 100 will now be explained with reference to the other figures.

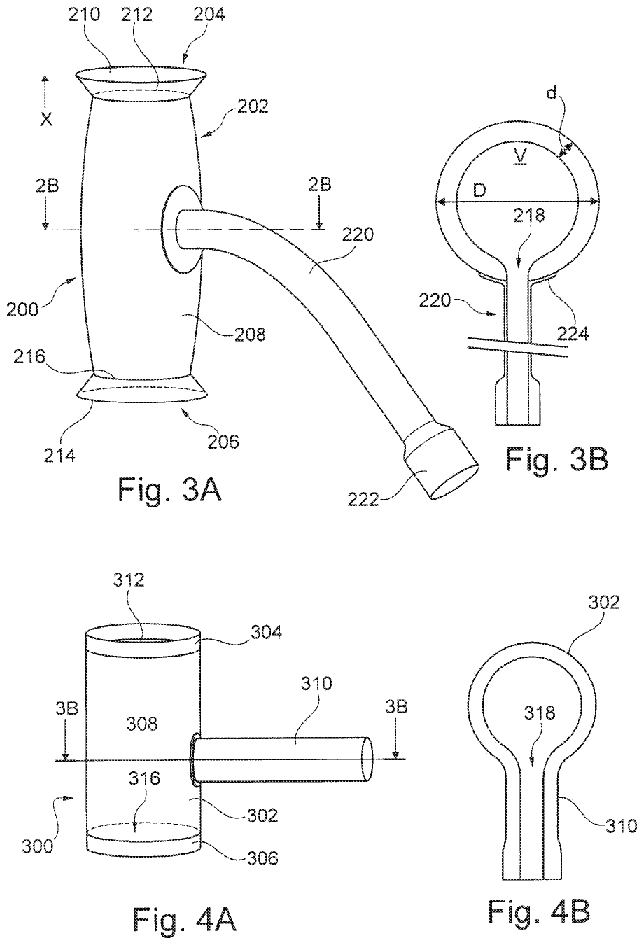

An embodiment of the device is shown in FIG. 3A in a longitudinal view and in FIG. 3B in a cross-section. The device 200 comprises a hollow body 202 which has a length L=8 cm in the x direction and a diameter D=3 cm. Here, the diameter firstly increases from the first end section 204 to the second end section 206, reaches a maximum in the middle of the intermediate section 208, and then decreases again. The hollow body thus has a bulged form. The hollow body itself is made from silicone. This has a wall thickness d of 3 mm. This wall thickness causes the cannula or the hollow body 202 to retain its form under simple pressure loads and thus ensures that the volume V provided inside the hollow body is available as blood reservoir.

The first and the second end section 204, 206 are constructed similarly. The components of the first end section will therefore be explained hereinafter representatively of the second section as well.

The first end section 204 comprises a collar 210, which is made of a thinned silicone, and for example can be sewn to an artificial tissue.

This artificial tissue can then be sewn to the SVC. As a result of the additional artificial tissue, different lengths can be taken into consideration when connecting the SVC and the IVC. A first opening 212 is situated within the first end section and blood can flow through said first opening into the intermediate section 208. Similarly to the collar 210, the second end section has a collar to hundred and 14 and a second opening 216. The material of the intermediate section 208 is selected such that its wall thickness is greater than the wall thickness of the material in the region of the first and second end section. It is hereby ensured that the intermediate section is more rigid than the first and second end section.

In the cross-section of FIG. 3B it can be seen that the hollow body 202 also comprises a third opening 218 and that a coupling device 220 is provided in the region of the opening 218, which coupling device is formed in the present exemplary embodiment as a silicone cannula. The silicone cannula 220 is connected in an integrally bonded manner to the hollow body 202. For example, the connection can be realised by means of an adhesive or by means of welding. However, a frictionally engaged connection, for example by sewing or stapling, is also possible. The silicone cannula 220 has a length of 40 cm and is configured such that the end 222 leading away from the hollow body 202 can be directly connected to an extracorporeal blood pump, for example the blood pump 108 of FIG. 2. The diameter of the cannula 220 can be selected such that it is suitable for the corresponding purpose. In other words, a cannula of smaller diameter can be selected in smaller children, and a cannula of larger diameter can be selected in larger children.

The cannula 220 has a flange 224 on the outer side of the hollow body 202, which flange serves as a reinforcement of the connection to the hollow body 202. An improved fastening between the cannula 220 and the hollow body 202 is hereby ensured.

It can be seen in the figures that the third opening is smaller than the first and second opening of the end sections. In the present exemplary embodiments of FIGS. 2-4, which each show a bulged convexity, the ratio of the flow area F_1 of the first opening 214 to the flow area F_3 of the third opening 218 is F_3/F_1≈0.5, i.e. the diameter of the third opening is approximately 7/10 of the diameter of the first opening. In other exemplary embodiments the ratio can be greater or smaller than that specified here. In the bulged exemplary embodiments described hereinafter the ratio of the flow areas can be similar to that described above.

A further embodiment of a device can be found in FIG. 4. The device 300 of FIG. 4A is shown in cross-section in FIG. 4B. The device 300 comprises a hollow body 302, which for example is made of a silicone of uniform cross-sectional diameter. The hollow body 302 also comprises a first end section 304 and a second end section 306, for which a tissue-like, but fluid-tight material has been selected. An intermediate section 308, in which a cannula 310 is directly integrated, is situated between the first end section 304 and the second end section 306. The intermediate section 308 and the cannula 310 are integrally connected to one another and are manufactured from a single piece. The first end section 304 and the second end section 306 are welded to the intermediate section 308 and can be directly sewn or stapled to vessel ends. At the end of the first end section 304 situated towards the intermediate section 308, there is an opening 312, which is the first opening. A corresponding second opening is shown schematically and provided with the reference sign 316. It can be seen in FIG. 4B that the cannula 310 attaches directly to the hollow body 302, wherein the lumen of the cannula 310 in the region of the third opening 318 opens out into the cavity of the hollow body 302.

The geometric form of the hollow body shown in FIG. 4 with uniform diameter can also be used for example in the embodiment of FIG. 3A. Likewise, the embodiment of the end sections as tissue materials that are welded to the intermediate section 308 can also be used in the device shown in FIG. 3, and similarly the end sections from FIG. 3 can also be used in the embodiment according to FIG. 4.

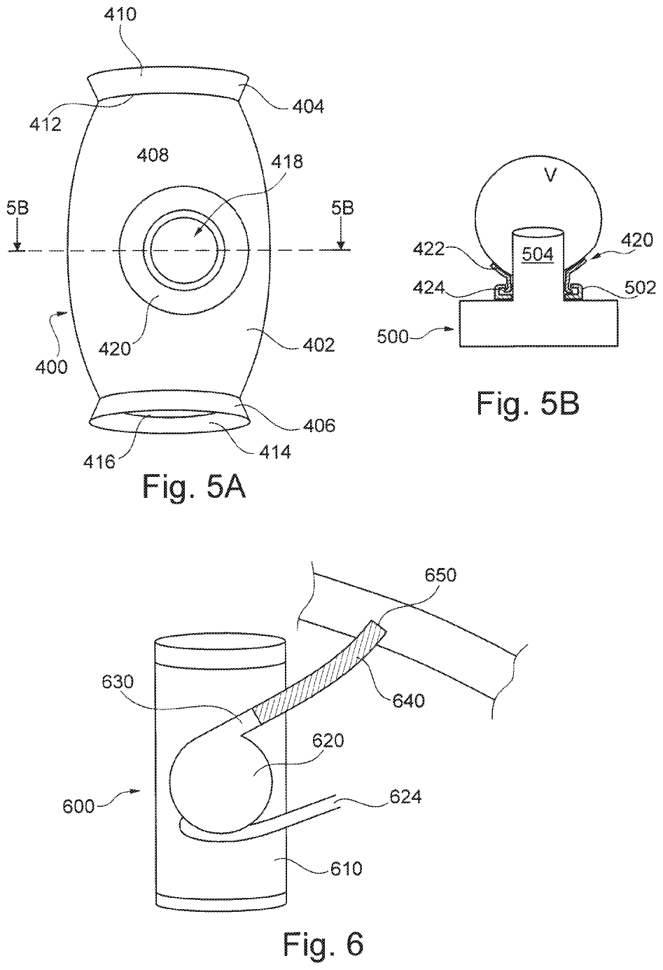

A further embodiment of a device is shown in FIG. 5. Here, FIG. 5B is a cross-section in the region of the third opening of the device shown in FIG. 5A. The device 400, as in the previous exemplary embodiments, comprises a hollow body 402, which has a first end section 404 and a second end section 406. An intermediate section 408 is situated between these two end sections. In the present exemplary embodiment the intermediate section 408 can be made from a biocompatible plastic or a silicone. The first and the second end section 404, 406 are welded to the intermediate section 408 and comprise a material that can be sewn to a vessel end. In this way, the vessel ends, for example the vessel ends of the SVC or IVC, can be sewn directly to the device. In addition, a “suture ring” is arranged in the intermediate section 408 and either can be directly coupled to a pump or can be attached to the cannula, which in turn can be coupled to a blood pump. The suture ring thus represents merely a specific embodiment of an adapter with which the device can be coupled to a blood pump directly or can be coupled to a blood pump indirectly via a further cannula.

The first end section, besides the collar 410, also comprises an opening 412. Similarly, the second end section comprises a collar 414 and an opening 416. The collar 410 or 414 has a width here of 2 cm and can be shortened once the collar has been fastened to the vessel end. The surgeon is hereby given the option to trim the end sections to size when the device is actually being implanted, without having to press the device onto the vessel by additional sections.

The intermediate section 408 comprises a third opening 418, which is surrounded by the suture ring 420. FIG. 5B shows the suture ring 420 in cross-section. The suture ring 420 comprises a collar 422 and a further collar 424, which is to be engaged from behind by a pump and which points radially outwardly from the hollow body. This “suture” ring, since the device has been provided with a hole, can be applied subsequently and either welded or adhesively bonded to the hollow body or connected by means of a suture. Alternatively, the suture ring can also be formed as an adapter, onto which a cannula, for example the cannula 114 or 104, can be fitted. To this end, the adapter can comprise for example a male part of a Luer lock branching radially away from the intermediate section, and a cannula having a female Luer lock can be fitted onto said male part. However, other plug-in systems that are conventional in the clinical field are also possible for connection of an adapter to a cannula for use with the device.

In the embodiment shown here the suture ring 420 is configured such that it can be engaged from behind by a pump 500. To this end the pump for example can comprise a mechanism 502 which enables permanent engagement of the collar 424 from behind. The inlet 504 of the pump, as shown in FIG. 5B, is fitted directly into the third opening 418, so that the entry of the inlet 504 comes to lie in the volume V of the hollow body 402. If blood now flows from the SVC and the IVC into the intermediate section, this is drawn into the pump through the inlet 504 of the pump, is acted on by force, and is expelled through the outlet 506. This intracorporeal heart assist pump can be for example an axial system or applying an axial force to the blood or can be a radial system for applying a radial force to the blood. The device can also be provided initially with only one opening 418 and, depending on the application, can be equipped with a corresponding adapter only just before the intervention. Adapters delivered with heart assist systems, for example the suture rings thereof, can be used here.

A system 600 formed of a device 610 and a blood pump 620 will be illustrated with reference to FIG. 6. The blood pump 620 is coupled here to the device 610 as shown in FIG. 5B. The blood pump 620 has a drive which is supplied with power by means of the cable 624. The cable can be guided to a transcutaneously implanted power supply system or can be guided outwardly through the skin of the patient so as to be connected there to a power source. The outlet 630 of the pump is connected to an outlet graft 640, which at its end 650 opposite the outlet 630 is coupled to the pulmonary artery. Here, an existing Fontan graft or Fontan tunnel can be used, for example, and this can be coupled to the outlet of the pump.

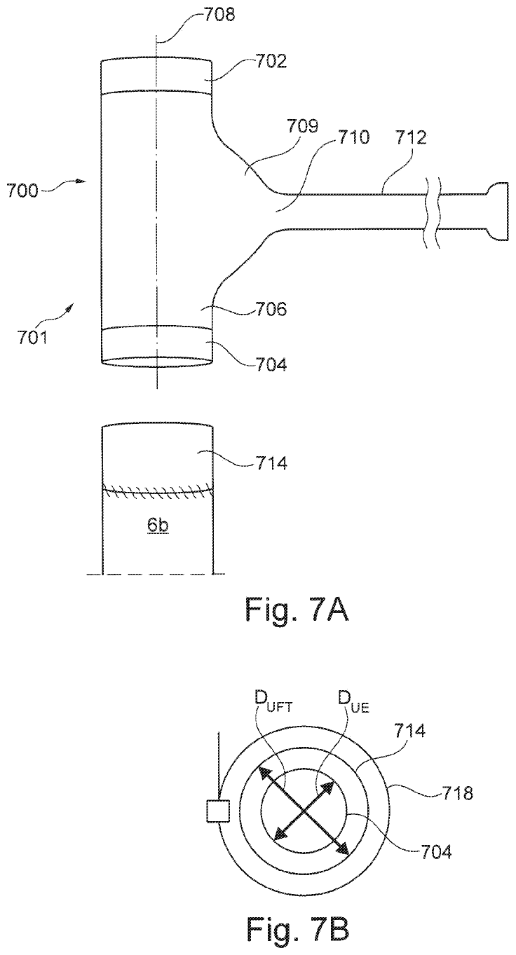

A further embodiment of a device and a method according to the application will be illustrated and explained with reference to FIGS. 7A and 7B.

The device 700 comprises a hollow body 701 with an upper end section 702, a lower end section 704 and an intermediate section 706 in-between. The intermediate section has an asymmetrical cross-section, wherein the area located on the right next to the body axis 708 firstly enlarges from the upper end section to the lower end section and then decreases again, whereas the area located to the left of the body axis remains substantially constant. This bulged section 709 is therefore not provided on both sides of the body axis 708, but merely on one side. An asymmetric cross-section can be understood generally to mean a cross-section which has a constant radius along a body axis at least over an angular range between 30° and 180°.

In addition, the wall thickness of the hollow body in the region of the largest cross-section, i.e. in the region of the third opening 710, where the coupling device 712 attaches, can be reduced in order to achieve an improved compliance, for example in the region of the right atrium. Alternatively to the coupling device, the third opening can also comprise a connector for a blood pump.

The IVC 6b is also provided with a lower edge 714 of the Fontan tunnel, i.e. the Fontan tunnel has not been completely removed, and instead the edge has been left in place in order to eradicate the need to form a further suture in the IVC, thus further relieving the load on the patient. Although not shown in the figures,—alternatively or additionally to the lower edge—an upper edge of the Fontan tunnel, which is sewn to the pulmonary artery, can remain in the body. In this way, the attachment of the device to the pulmonary artery or the upper edge of the Fontan tunnel is also simplified. To this end, the Fontan tunnel 7 is severed in such a way that an upper and/or lower edge, for example with a width between 0.5 cm and 3 cm, remain/remains connected to the IVC or pulmonary artery, such that there is no need to form new additional sutures in the body's own tissue.

In the present example the hollow body 701 is made from silicone at the upper end section and in the intermediate section 706. The lower end section 704, besides the silicone, also has a metallic reinforcement, which is arranged on the outer side 716 of the hollow body. The metallic reinforcement can be, for example, sintered titanium, which improves the in growth properties of the lower end section. The diameter DUE of the lower end section in the present exemplary embodiment is smaller than the diameter DUFT of the lower edge 714 of the Fontan tunnel 7. In this way, the lower end section can be introduced into the lower edge of the Fontan tunnel and then can be connected in a fluid-tight manner to a connection means, for example a cable tie 718.

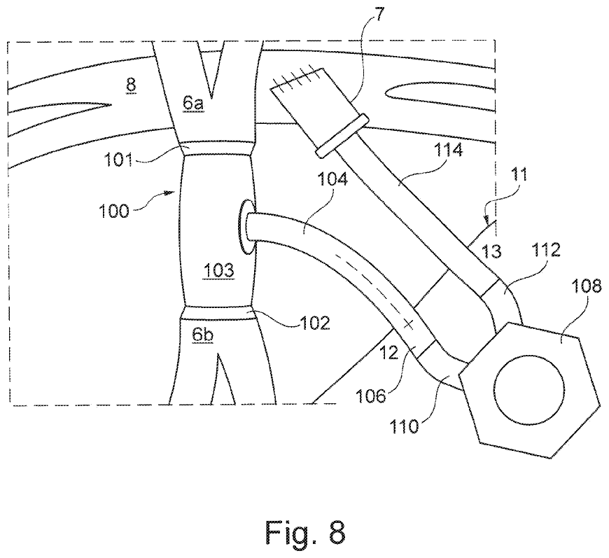

The method already discussed will be illustrated with reference to FIG. 8, in which the Fontan tunnel 7—in contrast to that shown in FIG. 2—is not removed, but instead is connected in its entirety or in shortened form to the further cannula 114. In this embodiment the cannula 114 does not have to be connected directly to the pulmonary artery 8, and therefore the pulmonary artery is not further loaded in the method proposed here.

The device proposed here can be formed for example by means of a casting process. However, it is also possible to produce the intermediate section or the entire hollow body by way of a three-dimensional printing method. In this way, the individual conditions within the patient body can be taken into consideration particularly efficiently.

Although, until now, the pump system has been described only as a transition system prior to a heart transplant, the pump system can also be used as a permanent heart assist system, if a suitable donor heart is not available. In this case the pump or the cannulas for attachment of the pump remains/remain permanently in the patient's body.

To clarify the use of and to hereby provide notice to the public, the phrases “at least one of <A>, <B>, . . . and <N>” or “at least one of <A>, <B>, <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, or N including any one element alone or the one element in combination with one or more of the other elements which may also include, in combination, additional elements not listed. Unless otherwise indicated or the context suggests otherwise, as used herein, “a” or “an” means “at least one” or “one or more.”

1. A device for connecting two blood vessel sections, the device comprising:
a fluid-tight hollow body with a first end section, a second end section and an intermediate section arranged between the first and second end section, the first end section having a first opening, the second end section having a second opening, and the intermediate section having a third opening and the first opening and the second opening each are connectable to a vessel section and thc intcrmcdiatc scction, in thc rcgion of thc third opcning, has
a coupling device for a blood pump or a blood-guiding cannula in a region of the third opening of the intermediate section.

2. The device according to claim 1, wherein the third opening is smaller than the first and/or second opening.

3. The device according to claim 1, wherein the coupling device is integrally connected to the intermediate section.

4. The device according to claim 1, wherein the coupling device is a cannula.

5. The device according to claim 1, wherein the coupling device is a suture ring.

6. The device according to claim 1, wherein the coupling device is an adapter for attachment of a pump or a blood-guiding cannula.

7. The device according to claim 1, wherein the first and second end section are formed in such a way that they can be connected in a fluid-tight manner to a blood vessel.

8. The device according to claim 7, wherein the first and/or second end section comprise/comprises a collar which is made of a textile material or a material such that the collar can be shortened.

9. The device according to claim 1, wherein the hollow body is formed in one piece.

10. The device according to claim 1, wherein the hollow body has a cross-section which increases from the first end section to the intermediate section and decreases again from the intermediate section to the second end section.

11. The device according to claim 1, wherein the hollow body has a length between the first and second end section between 2 and 15 cm.

12. The device according to claim 1, wherein the blood vessel sections to be connected are the superior vena cava and the inferior vena cava.

13. A system comprising:
a blood pump and
a device for connecting two blood vessel sections, the device including a fluid-tight hollow body with a first end section, a second end section and an intermediate section arranged between the first and second end section, the first end section having a first opening, the second end section having a second opening, and the intermediate section having a third opening and the first and second opening each are connectable to a vessel section and a coupling device for a blood pump or a blood-guiding cannula in the region of the third opening of the intermediate section.

14. The system according to claim 13, wherein the blood pump is an extracorporeal or intracorporeal heart assist pump.

15. (canceled)

16. A method for implanting a device, the implantable device including a device for connecting two blood vessel sections, the device including a fluid-tight hollow body with a first end section, a second end section and an intermediate section arranged between the first and second end section, the first end section having a first opening, the second end section having a second opening, and the intermediate section having a third opening and the first and second opening each are connectable to a vessel section and a coupling device for a blood pump or a blood-guiding cannula in the region of the third opening of the intermediate section, the method comprising:
connecting the first end section to the superior vena cava and the second end section to the inferior vena cava,
coupling the coupling device to an input of a blood pump, and
connecting an output of the blood pump to a pulmonary artery.

17. The method according to claim 16, wherein the blood pump is an extracorporeal heart pump and is connected to a cannula extending from a body between the third opening and the input of the blood pump, and the output of the blood pump is coupled by means of a further cannula extending from a pulmonary artery to the output.

18. The method according to claim 16, wherein the blood pump is an intracorporeal heart pump and the coupling device is a coupling ring which is fixedly connected to the hollow body

19. The device according to claim 1, wherein the coupling device is a silicone cannula

20. The device according to claim 1, wherein the device relieves the heart of a patient with failing Fontan circulation.
